(19) United States
(12) Patent Application Publication
(10) Pub. No.: US 2009/0048609 A1
(43) Pub. Date: Feb. 19, 2009

(54) UTERINE CANNULA

(75) Inventors: William Atiomo, Nottingham (GB); David John Laird, Nottingham (GB)

Correspondence Address: DAVIS WRIGHT TREMAINE LLP/Los Angeles, 865 FIGUEROA STREET, SUITE 2400, LOS ANGELES, CA, 90017-2566, US

(73) Assignee: N.G.D. LIMITED, Nottingham (GB)

(21) Appl. No.: 11/913,926

(22) PCT Filed: May 11, 2006
(86) PCT No.: PCT/GB2006/001728

§ 371 Date: Aug. 13, 2008

(30) Foreign Application Priority Data
May 11, 2005 (GB) 0509565.8

(51) Int. Cl. A61B 17/42

(52) U.S. Cl. 606/119

(57) ABSTRACT

A uterine cannula having a proximal end and distal end and comprising: a hollow outer tube having a proximal end and a distal end; an inner tube receivable within the outer tube, and having a proximal end and a distal end, a distal portion of the inner tube extending beyond the distal end of the outer tube, and comprising one or more apertures, and a conduit extending along the inner tube; the inner tube being moveable within the outer tube, such that the length of the distal portion of the inner tube that extends beyond the distal end of the outer tube is adjustable, the cannula further comprising a seal positioned at, or towards the distal end of the cannula.

This invention relates to a uterine cannula for use in gynecological procedures, and particularly, but not exclusively to a uterine cannula for use in hydrotubation procedures, and for uterine manipulation during laparoscopic procedures. Such procedures may be used in the determination of causes of infertility in women.

Infertility is an upsetting problem that can affect as many as 1 in 6 couples in the United Kingdom.

There are various factors that lead to infertility in a couple. For example on the male side, there may be a low sperm count, poor sperm motility, or abnormal sperm shape. Female factors include ovulation failure, blocked fallopian tubes, endometriosis and uterine fibroids.

In order to determine which female factors if any, may be contributing to infertility in a couple, it is necessary for a woman to have the patency of her fallopian tubes checked as part of initial investigations.

It is known to check the patency of a woman's fallopian tubes by injecting a dye into a woman's uterus through her cervix. The flow of the dye is then checked as it passes through the uterus and fallopian tubes.

The passage of the dye through the uterus and fallopian tubes may be measured using non-invasive procedures such as by means of an ultrasound scan or by means of x-rays. Alternatively, laparoscopic procedures may be used in which a telescope is inserted through the umbilicus in order to visualise the pelvis while a dye test, known as hydrotubation, is carried out during which methylene blue dye is injected through the cervix.

It is known to carry out gynecological procedures for example, to check the patency of fallopian tubes using a device of the type described in U.S. Pat. No. 5,195,964.

A problem with a device of the type disclosed in U.S. Pat. No. 5,195,964 is that it is necessary to hold the device in place by means of forceps during a gynecological procedure. This may mean that a surgeon or other medical practitioner may have to use one hand to control the forceps and may thus have only one hand available for carrying out the procedure. Alternatively, it may be necessary for an assistant to hold the forceps, thus “crowding” the area in which the surgeon is to operate.

The device disclosed in U.S. Pat. No. 5,195,964 comprises an elongate portion that, in use, extends into the uterus of the patient. This portion allows uterine manipulation to take place.

Another problem with the device of the type shown in U.S. Pat. No. 5,195,964 is that the length of the portion that extends into the uterus of a patient is not adjustable, and therefore it is not possible to adjust the device to take into account different sizes of uterus.

According to a first aspect of the present invention there is provided a uterine cannula having a proximal end and a distal end and comprising:
a hollow outer tube having a proximal end and a distal end;
an inner tube receivable within the outer tube, and having a proximal end and a distal end, a distal portion of the inner tube extending beyond the distal end of the outer tube, and comprising one or more apertures, and a conduit extending axially along the inner tube; the inner tube being moveable within the outer tube, such that the length of the distal portion of the inner tube that extends beyond the distal end of the outer tube is adjustable,
the cannula further comprising a seal positioned at, or towards the distal end of the cannula.

By means of the present invention, a fluid such as a dye may be injected into a patient's uterus by injecting the dye into the conduit formed in the inner tube of the uterine cannula.

The one or more apertures formed in the distal portion of the inner tube result in the distal portion being in fluid communication with the conduit. This means that dye injected into the conduit will flow into the uterus via the one or more apertures.

The distal portion of the inner tube allows for uterine manipulation during any gynecological procedure. In other words the distal portion of the inner tube enables a surgeon or other medical personnel to manipulate the uterus and inspect the pelvis during a procedure.

The inner tube may be connected to the outer tube by means of a connector.

The distal portion of the inner tube may be co-linear with the remainder of the inner tube, or it may be angled relative to the remainder of the inner tube.

The cannula may further comprise an indicator for indicating to a user the orientation of the distal end of the inner tube. This is particularly useful when the distal portion is angled relative to the remainder of the inner tube.

Because the inner tube is movable within the outer tube, the length of the distal portion of the inner tube that extends into the uterus of a patient may be adjusted to fit the particular dimensions of the uterus in which the cannula has been placed. Such adjustment of the length of the distal portion of the inner tube reduces the chances that the uterus will be perforated during use of the device.

The inner tube is conveniently movable axially within the outer tube.

Because a conduit is formed in the inner tube, dye may be readily injected into the inner tube, contained within the inner tube, to then emerge from the inner tube via the one or more apertures formed in the distal portion of the inner tube. This allows dye to be readily contained within the inner tube, thus reducing the possibility that dye will leak inappropriately from the cannula.

The cannula may comprise a single aperture at its distal end. By means of the single aperture dye may in use, be directed into the fallopian tubes of a patient. Because the single aperture is formed at the distal end of the inner tube, preferably at a distal tip of the inner tube, pressure generated within the cannula by an injector such as a syringe used to inject dye into the cannula, will cause dye to be ejected from the single aperture with some force. This allows the dye to reach the fallopian tubes.

Preferably, the conduit extends along the entire length of the inner tube, although in some embodiments, it may extend only partially along the length of the inner tube.

Advantageously, the cannula further comprises a lock having an unlocked state in which the inner tube may move relative to the outer tube, and a locked state in which the position of the inner tube is fixed relative to the outer tube.

Preferably, the lock comprises a tubular portion adapted to be positioned around the inner tube of the cannula. The tubular portion comprising an aperture adapted to receive a screw for locking the tubular portion in place on the inner tube.

By means of the lock, once the length of the distal portion of the inner tube has been adjusted to fit the uterus on which a procedure is to be carried out, by moving the inner tube relative to the outer tube, the relative positions of the inner tube and the outer tube may be fixed by moving the lock into its locked state from the unlocked state.

The lock may be positioned at any convenient point on the cannula, but preferably it is positioned on the inner tube. The lock may be positioned at any point along the inner tube, but preferably is positioned towards the proximal end of the inner tube.

Advantageously, the seal is positioned on the outer tube at or towards the distal end of the outer tube, although it could be placed at any other convenient position. The seal is preferably substantially cone shaped in order to allow easy insertion into the cervix of a patient.

Advantageously, the seal is threaded on an outer surface. This means that in use, a seal may be formed between the cannula and the cervix of a patient, by rotating the seal relative to the cervix thus screwing the seal into the wall of the cervix.

An advantage of having a threaded seal, is that once a seal has been formed by rotating the seal within the cervix, the seal may be maintained without a surgeon or other medial personnel having to apply force to the seal, for example through the use of forceps.

Conveniently, the seal comprises a broken thread. The thread may be broken at one or more points along the thread. This means that there is no continuous path along which dye that has been injected into the uterus can run, thus reducing the possibility that dye will leak from the uterus during use.

In addition, the broken thread of the seal minimises cervical trauma caused by use of the cannula.

Preferably, the cannula further comprises a first handle positioned at or towards the proximate end of the inner tube. The first handle is positioned appropriately for a surgeon or other medical personnel to grip the handle during use of the cannula. In addition, the first handle allows the cannula to be rotated thus causing the seal to be screwed into the cervix of the patient.

Preferably, the first handle comprises an aperture allowing access to the inner conduit of the inner tube. This means that, when it is necessary to inject dye into the uterus, an injector may be inserted into the aperture in the first handle, such that the injector is in fluid communication with the conduit of the inner tube. A dye injected into the conduit via the aperture in the first handle may thus be injected into the uterus, since dye inserted in the conduit will exit the inner tube via the one or more apertures formed in the distal portion of the inner tube.

Preferably, the cannula further comprises a second handle positioned on the outer tube at, or towards the proximate end of the outer tube.

Advantageously, the second handle comprises an aperture shaped to receive the inner tube thus allowing the inner tube to be inserted within the outer tube.

Preferably, the cannula further comprises a clip that is moveable along the outer tube of the cannula. The clip is preferably slideably moveable along the outer tube, and may be locked at a predetermined position on the outer tube. The clip is shaped to receive forceps which may be used during use of the cannula.

According to a second aspect of the present invention there is provided a method for carrying out a gynecological procedure using a uterine cannula according to the first aspect of the present invention.

Preferably the method for carrying out a gynecological procedure according to the second aspect of the present invention, comprises the steps of:
determining the length of the uterine cavity of a patient;
inserting the cannula into the uterus of the patient via a cervix of the patient such that the length of the inner tube within the uterus is less than the length of the uterus;
adjusting the seal to form a loose fit in the cervix of the patient;
twisting the seal to form a substantially fluid-tight seal between the cervix and the seal;
injecting dye into the inner tube and causing the dye to be ejected through the one or more apertures formed in the distal end of the inner tube.

The invention will now be further described by way of example only with reference to the accompanying drawings in which:

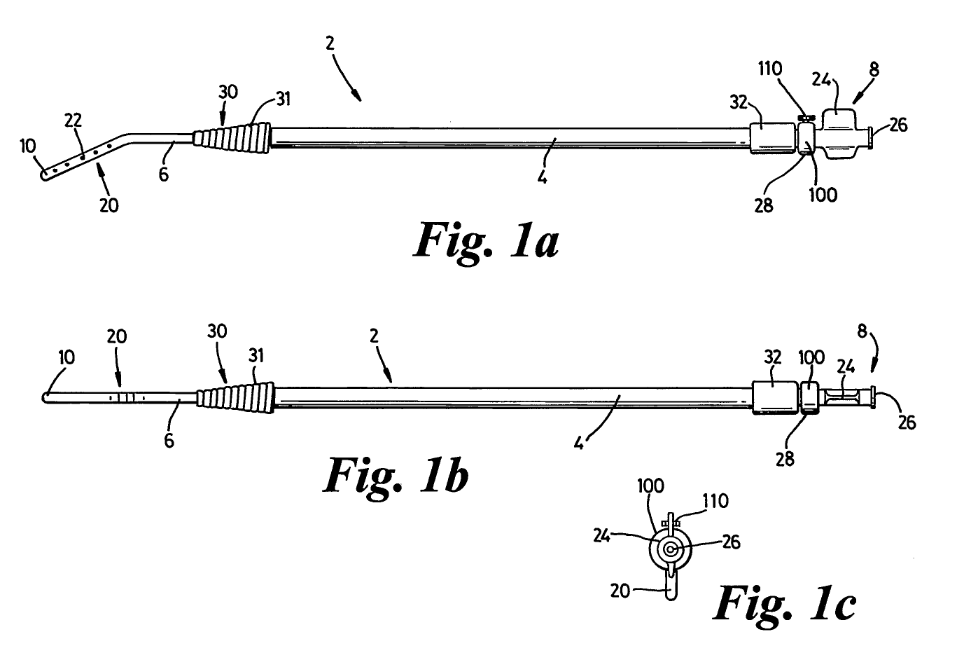

FIGS. 1a, 1b, and 1c are schematic representations of a uterine cannula according to the present invention;

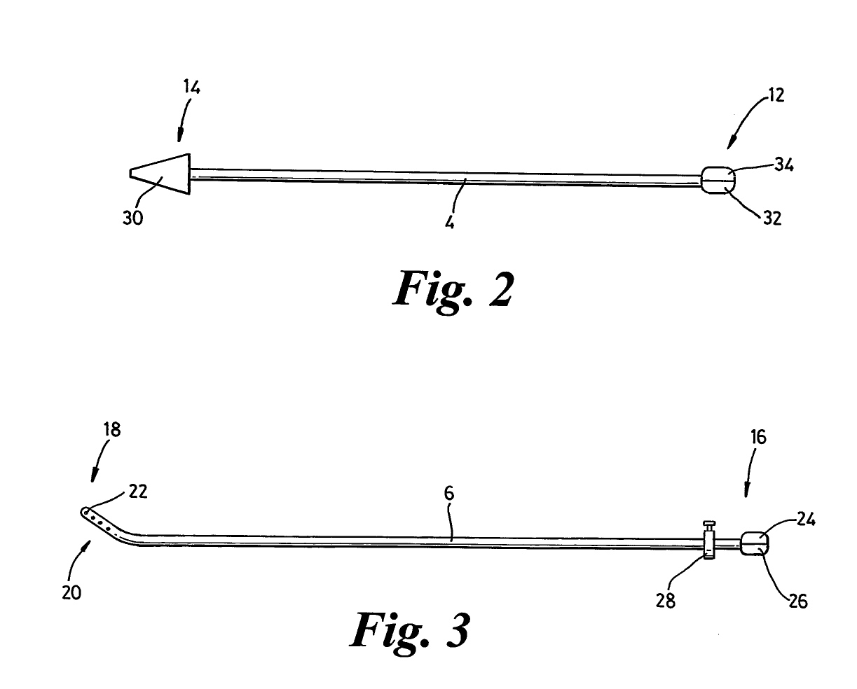

FIG. 2 is a schematic representation of the outer tube forming part of the uterine cannula of FIG. 1; and

FIG. 3 is a schematic representation of the inner tube forming part of the uterine cannula shown in FIG. 1.

Referring to the figures, a uterine cannula according to the present invention is designated generally by the reference numeral 2.

In FIG. 1a the uterine cannula 2 is in a first position, in FIG. 1b the uterine cannula has been rotated through approximately 900 to a second position, and FIG. 1c is a cross-sectional representation of the uterine cannula 2 as shown in FIG. 1a.

The cannula 2 comprises a hollow outer tube 4, and a hollow inner tube 6. The cannula has a proximate end 8 and a distal end 10. Similarly, the outer tube 4 comprises a proximal end 12 and a distal end 14, and the inner tube 6 comprises a proximate end 16 and a distal end 18.

The inner tube comprises a first handle 24 comprising an aperture 26.

The inner tube further comprises a locking device 28.

The inner tube 6 comprises a distal portion 20 comprising a plurality of perforations 22. In this embodiment the distal portion 20 is angled relative to the remainder of the inner tube 6. The cannula further comprises an indicator (not shown) for indicating the orientation of the distal portion of the inner tube relative to the remainder of the inner tube. The means for determining the orientation may be, for example, a directional arrow placed on the handle 24 of the inner tube.

The outer tube comprises a seal 30 positioned at the distal end 14 of the outer tube 4. In this embodiment the seal 30 is substantially cone shaped and has a threaded outer surface 31.

The outer tube further comprises a second handle 32 which also comprises an aperture 34.

The cannula 2 is assembled by inserting the distal end 18 of the inner tube 6 into the outer tube 4 via the aperture 34 formed in the second handle 32.

The locking device 28 has an unlocked state in which the inner tube 6 is movable within the outer tube 4, and a locked stated in which the relative positions of the inner tube 6 and the outer tube 4 are locked in position.

Before carrying out a procedure, a surgeon or other medical personnel establishes the dimensions of the uterus of a patient upon whom a procedure is to be carried out. This is achieved through use of a measuring device such as a uterine sound. The surgeon then moves the inner tube relative to the outer tube until the length of the distal portion 20 of the inner tube that extends beyond distal end 14 of the outer tube is appropriate for the dimensions of the uterus to be examined. The locking device is then moved into its locked positioned so that the inner tube 6 and the outer tube 4 are held in this position. This means that during the procedure to be carried out, the length of the distal portion 20 extending beyond the distal end 14 of the outer tube will remain fixed during the procedure. This reduces the possibility that the uterus will become perforated during any procedure carried out using the cannula 2.

The lock preferably comprises a tubular portion 100 adapted to be positioned around the inner tube at a point distal to the handle of the outer tube. The lock further comprises a screw 110 receivable within an aperture formed within the tubular section. When a screw is screwed through the aperture it makes contact with the inner tube, and locks the inner tube and the outer tube together in order that they are fixed relative to one another.

In order to carry out a hydrotubation procedure on a patient, the assembled cannula 2 is inserted into the uterus of a patient via the patient's cervix. The uterus is inserted such that the length of the inner tube within the uterus of the patient is just less than the length of the uterus as measured by the uterine sound. Preferably, the cannula is positioned within the uterus so that there is a gap of at least 0.5 cm between a distal end of the inner tube, and a distal wall of the uterus. When the cannula 2 is in place, the seal 30 will be positioned within the cervix. In order to form a seal between the cannula 2 and the cervix, the cannula is rotated using handle 26, thus causing the threaded seal 30 to rotate and to be screwed into the cervix thus forming a fluid tight seal between the cervix and the seal. Once the seal has been formed, it is not necessary for a surgeon to apply a force on the seal to maintain the seal.

The thread formed on the seal 30 is preferably a broken thread. This means that there is no continuous path along which dye injected into the uterus may travel and thus leak from the cannula via the thread of the seal. This minimises the amount of dye leaking from the uterus during use of the cannula.

The broken thread also reduces trauma caused to the cervix through use of the cannula.

Further, the grooves of the cone are shallow in order to minimise cervical trauma and to reduce the side of the track through which dye could possibly travel in order to leak out of the uterus.

Preferably, the seal is made from a plastics material but could also be made from stainless steel or any other suitable material. When the seal is made from a plastics material, it is made by injection moulding. The base of the seal will be approximately 15 mm, and the seal will be approximately 35 mm long. The threads may have any convenient dimensions and may for example, be spaced apart by 3 mm and may be approximately 1 mm deep.

For the avoidance of doubt, the seal and threads could be any suitable dimensions.

In order to inject dye into a uterus, an injector is attached to the aperture 26 formed in the first handle. Dye may then be injected into the inner conduit (not shown) of the inner tube 6 via the aperture 26, to emerge via the perforations 22 formed in the distal portion 20 of the inner tube 6.

Due to the fluid tight seal formed between the cervix and the seal 30, dye injected in to the uterus will remain in the uterus until the procedure has been completed.

The distal portion 20 not only allows dye to be injected into the uterus, but also allows a surgeon to manipulate the uterus in order to, for example inspect the pelvis.

The present invention thus provides a uterine cannula having a distal portion allowing uterine manipulation to take place. The length of the distal portion is adjustable to take into account the size of the patient's uterus. In addition, the uterine cannula comprises a seal in the form of a threaded cone, which provides a fluid tight seal within the cervical canal of a patient.

1. A uterine cannula having a proximal end and distal end and comprising:
a hollow outer tube having a proximal end and a distal end;
an inner tube receivable within the outer tube, and having a proximal end and a distal end, a distal portion of the inner tube extending beyond the distal end of the outer tube, and comprising one or more apertures, and a conduit extending along the inner tube; the inner tube being moveable within the outer tube, such that the length of the distal portion of the inner tube that extends beyond the distal end of the outer tube is adjustable,
the cannula further comprising a seal positioned at, or towards the distal end of the cannula.

2. A uterine cannula as claimed in claim 1 further comprising an indicator for indicating the orientation of the distal inner tube during use of the cannula.

3. A uterine cannula as claimed in claim 1, further comprising a lock having an unlocked state in which the inner tube may move relative to the outer tube, and a locked state, in which the position of the inner tube is fixed relative to the outer tube.

4. A uterine cannula as claimed in claim 3, wherein the lock is positioned on the inner tube.

5. A uterine cannula as claimed in claim 1, wherein the seal is positioned on the outer tube at or towards the distal end of the outer tube.

6. A uterine cannula as claimed in claim 5 wherein the seal is substantially cone shaped.

7. A uterine cannula as claimed in claim 1, wherein the seal is threaded on an outer surface of the seal.

8. A uterine cannula as claimed in claim 7 wherein the seal comprises a broken thread.

9. A uterine cannula as claimed in claim 1, further comprising a first handle positioned at, or towards the proximate end of the inner tube.

10. A uterine cannula as claimed in claim 9 wherein the first handle includes an aperture.

11. A uterine cannula as claimed in claim 1, further comprising a second handle positioned on the outer tube at, or towards the proximate end of the outer tube.

12. A uterine cannula as claimed in claim 1, further comprising a clip.

13. A uterine cannula as claimed in claim 12 wherein the clip is slideably moveable along the outer tube.

14. A method of carrying out a gynecological procedure using a uterine cannula as claimed in claim 1.

15. A method as claimed in claim 14 comprising the steps of:
determining the length of the uterine cavity of a patient;
inserting the cannula into the uterus of the patient via a cervix of the patient such that the length of the inner tube within the uterus is less than the length of the uterus;
adjusting the seal to form a loose fit in the cervix of the patient;
twisting the seal to form a substantially fluid-tight seal between the cervix and the seal;
injecting dye into the inner tube and causing the dye to be ejected though the one or more apertures formed in the distal end of the inner tube.

16-17. (canceled)
